(19) United States
(12) Patent Application Publication
(10) Pub. No.: US 2003/0195630 A1
(43) Pub. Date: Oct. 16, 2003

(54) Disc augmentation using materials that expand in situ

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/411,495

(22) Filed: Apr. 10, 2003

Related U.S. Application Data
(60) Provisional application No. 60/371,546, filed on Apr. 10, 2002.

(51) Int. Cl. A61F 2/44

(52) U.S. Cl. 623/17.16

(57) ABSTRACT

A method of augmenting a nucleus pulposus within an annulus fibrosis. A material having a relatively thin, elongated first state is inserted through the annulus, after which it expands or otherwise assumes a shape that is more rounded when implanted. In the preferred embodiment, for introduction the material is relatively rigid or hard and relatively thin, resembling a needle or a nail. The size, shape, and consistency of the material allow the device to be pushed through the fibers of the annulus fibrosis, preferably without an incision, and into the nucleus pulposus and/or disc space. The resultant shape assists the nucleus pulposis in acting as a “shock absorber,” and the expansion of the material also makes extrusion unlikely. Various materials qualify for this purpose according to the invention. Materials that change shape with temperature include memory-effect alloys such as Nitinol and substances such as stearle methacrylate. Materials that change in shape in the presence of moisture include hydrogels and other substances that imbibe water. Materials that expand due to chemical reaction include various foams, and the like, some of which may be applied in two-part form.

### REFERENCE TO RELATED APPLICATION

This application claims priority from U.S. Provisional Patent Application Serial No. 60/371,546, filed Apr. 10, 2002, the entire content of which is incorporated herein by reference.

### FIELD OF THE INVENTION

This invention relates generally to spinal surgery and, in particular, to disc augmentation using materials that expand in situ, including shape-memory materials.

### BACKGROUND OF THE INVENTION

Several hundred thousand patients undergo intervertebral disc operations each year. In the case of a healthy anatomy, the nucleus pulposus is entirely surrounded by the annulus fibrosis. In the case of a herniated disc, however, a portion of the nucleus pulposus has ruptured through a defect in the annulus fibrosis. Following a partial discectomy to treat the condition, a space remains adjacent a hole or defect in the annulus fibrosis following removal of the disc material. Some type of artificial disc replacement device is typically used to fill this void.

Numerous artificial disc replacement devices have been described, some using materials with shape-memory properties. Alternatively, dehydrated hydrogels can be placed into the disc space. Once inside the disc, the dehydrated hydrogel imbibe fluids and swell to a desired shape. A hole must be cut into the annulus fibrosis to insert the dehydrated hydrogel. Unfortunately, the hydrogel devices frequently extrude through the hole in the annulus fibrosis.

### SUMMARY OF THE INVENTION

This invention resides in a method of augmenting a nucleus pulposus within an annulus fibrosis. According to the preferred embodiment, a material having a relatively thin, elongated first state is inserted through the annulus, after which it expands or otherwise assumes a shape that is more rounded when implanted. The resultant shape assists the nucleus pulposis in acting as a “shock absorber,” and the expansion of the material also makes extrusion unlikely.

Various materials qualify for this purpose according to the invention. Materials that change shape with temperature include memory-effect alloys such as Nitinol and substances such as stearle methacrylate. Materials that change in shape in the presence of moisture include hydrogels and other substances that imbibe water. Materials that expand due to chemical reaction include various foams, and the like, some of which may be applied in two-part form.

In the preferred embodiment, for introduction the material is relatively rigid or hard and relatively thin, resembling a needle or a nail. The size, shape, and consistency of the material allow the device to be pushed through the fibers of the annulus fibrosis, preferably without an incision, and into the nucleus pulposus and/or disc space.

### BRIEF DESCRIPTION OF THE DRAWINGS

FIG. 1 illustrates a shape-memory material according to the invention in a first form which is hard and thin, and resembles a needle or a nail;

FIG. 2 illustrates the material of FIG. 1 assuming a second, expanded form once inside a disc;

FIG. 3 shows how a first group of devices which become spherical may be followed by a subsequent group of the same or different terminal shapes;

FIG. 4 shows how multiple groups fill voids or interstitial areas with the disc space;

FIG. 5 shows an axial cross section of the disc;

FIG. 6A shows an axial cross section of a disc and an alternative embodiment of the invention;

FIG. 6B an axial cross section of a disc and coiled wires shown in FIG. 6A;

FIG. 6C is an axial cross section of a disc and an introducer for the coiled wires shown in FIG. 6A;

FIG. 7A shows a view of the side of a coiled wire in an extended form;

FIG. 7B is a view of the side of the coiled wire drawn in FIG. 7A; and

FIG. 7C shows a view of the side of an alternative embodiment of the coiled wires.

### DETAILED DESCRIPTION OF THE INVENTION

The disc augmentation devices and method described herein take advantage of materials that change shape and/or consistency when placed into a disc. Various materials qualify for this purpose according to the invention. Materials that change shape with temperature include memory-effect alloys such as Nitinol and substances such as stearle methacrylate. Materials that change in shape in the presence of moisture include hydrogels and other substances that imbibe water. Materials that expand due to chemical reaction include various foams, and the like, some of which may be applied in two-part form.

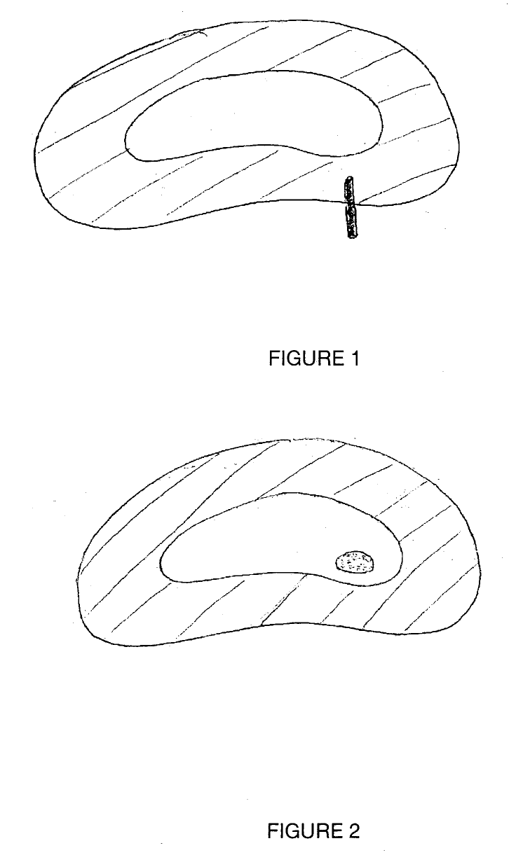

In the preferred embodiment, for introduction the material is relatively rigid or hard and relatively thin, resembling a needle or a nail. The size, shape, and consistency of the material allow the device to be pushed through the fibers of the annulus fibrosis, preferably without an incision, and into the nucleus pulposus and/or disc space, as shown in FIG. 1.

Once inside the disc space or nucleus, the material assumes a different shape when exposed to body temperature, moisture, and/or biological constituents. Other environmental changes could be used to convert the material from one form to another form. In the preferred embodiment, the material expands into compressible and/or resilient spherical shape, as shown in FIG. 2. If a shape-memory wire is used it may resultant shape assists the nucleus pulposis in acting as a “shock absorber.” The expansion of the material also makes extrusion unlikely.

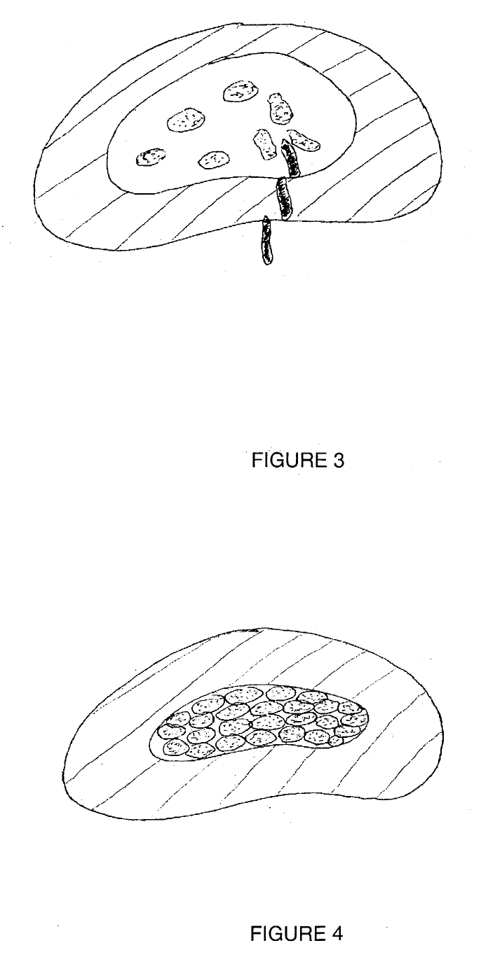

Multiple devices may also be placed into a single disc according to the invention. For example, as shown in FIG. 3, a first group of devices which expand may be followed by one or more subsequent groups of the same or different terminal shapes to fill voids or interstitial areas (FIG. 4).

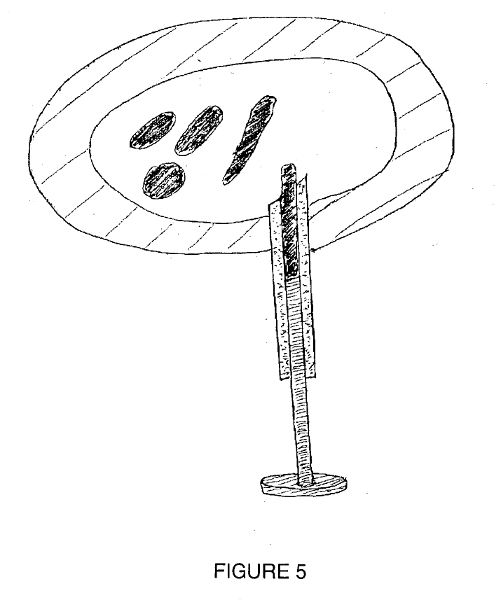

FIG. 5 is an axial cross section of the disc, the disc augmentation devices drawn in FIG. 3, and an optional instrument to insert the disc augmentation devices. The needle-like device is placed through the annulus fibrosis (AF). The disc augmentation devices are inserted through the cannula of the instrument. A plunger-like component pushes the disc augmentation devices into the nucleus pulposus (NP). The instrument avoids multiple holes that may result from inserting multiple disc augmentation devices. The instrument also aids the insertion of disc augmentation devices that are not strong enough to force the devices through the AF.

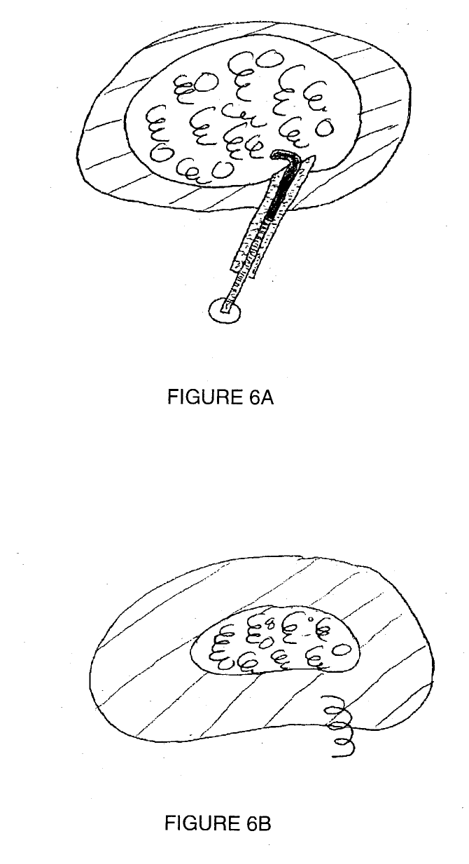

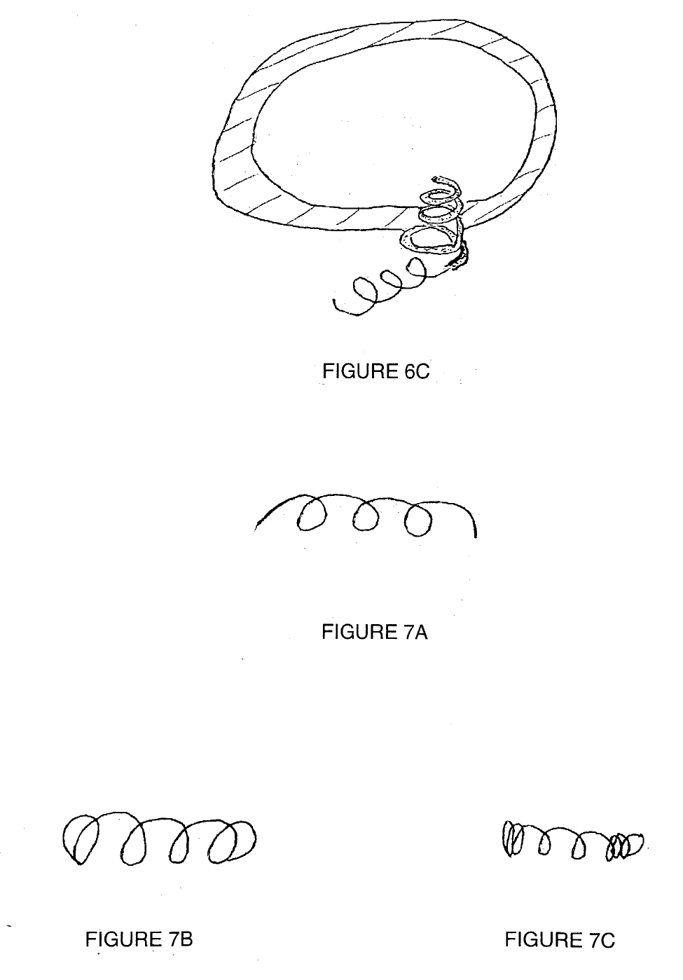

FIG. 6A is an axial cross section of the disc and an alternative embodiment of the invention related to FIG. 11 of co-pending U.S. patent application Ser. No. 09/807,820, incorporated by reference, wherein the wires are inserted through a hole created in the AF after removing at least a portion of the NP. In contrast, the instant invention preferably inserts one or more coiled wires into the disc space without removing any NP. The coiled wires straighten as they pass through the cannula. Once inside the disc space the wires resume their coiled shape. Coils made of polymers could also be used.

FIG. 6B is an axial cross section of the disc and the coiled wires drawn in FIG. 6A. The wires are twisted through the AF. FIG. 6C is an axial cross section of the disc and the coiled wires drawn in FIG. 6A. The coiled wires are inserted through a coiled cannula.

FIG. 7A is a view of the side of a coiled wire drawn in FIG. 6A. The coiled wire is drawn in an extended form. FIG. 7B is a view of the side of the coiled wire drawn in FIG. 7A. The coiled wire is drawn in its second, contracted shape. The ends of the wire return to a position near the adjacent coil. The second shape of the coiled wire is unlikely to work its way through the AF. The points or ends of the wires are not exposed in the second shape. Shape-memory or other appropriate materials could be used to achieve the “blunt” embodiment of the coils. The wires change shape after insertion in the disc space.

FIG. 7C is a view of the side of an alternative embodiment of the coiled wires. The coils at the ends of the wire become closer together in the second shape. Similar to the embodiment of the wires drawn in FIG. 7B, the second shape of the wire helps prevent the wires from migrating through the AF.

I claim:

1. A method of augmenting a nucleus pulposus within an annulus fibrosis, the method comprising the steps of:
providing a material having a relatively thin, elongated first state and a second state which is more rounded when implanted in, or adjacent to, an nucleus pulposus; and
inserting the material through the annulus fibrosis in the first state so that it may expand into the second state once inside the disc.

2. The method of claim 1, wherein the material expands due to a change in temperature.

3. The method of claim 1, wherein the material expands through exposure to moisture.

4. The method of claim 1, wherein the material expands through exposure to one or more chemical or biological constituents.

5. The method of claim 1, wherein the material is inserted through the annulus fibrosis without an intentional incision.

6. The method of claim 1, wherein the second state is generally spherical.

7. The method of claim 1, wherein the second state is a wire ball.
